(19) United States
(12) Patent Application Publication
(10) Pub. No.: US 2015/0005591 A1
(43) Pub. Date: Jan. 1, 2015

(54) TISSUE TO END TIDAL CO2 MONITOR

(71) Applicant: The Regents of the University of California, Oakland, CA (US)

(72) Inventor: Daniel Davis, Cardiff, CA (US)

(73) Assignee: The Regents of the University of California, Oakland, CA (US)

(21) Appl. No.: 14/376,122

(22) PCT Filed: Jan. 30, 2013
(86) PCT No.: PCT/US13/23925

§ 371 Date: Jul. 31, 2014

Related U.S. Application Data
(60) Provisional application No. 61/593,170, filed on Jan. 31, 2012.

(51) Int. Cl. A61B 5/00; A61B 5/1455; A61B 5/083

(52) U.S. Cl. CPC A61B 5/7278; A61B 5/0836; A61B 5/14551; A61B 5/6819; A61B 5/682; A61B 5/6815; A61B 5/7282
USPC 600/301

(57) ABSTRACT

A method comprises measuring tissue carbon dioxide levels of a living organism, measuring end-tidal carbon dioxide levels of the living organism; and calculating at least one tissue-to-end-tidal carbon dioxide gradient value based on the measured tissue carbon dioxide levels and the measured end-tidal carbon dioxide levels, the tissue-to-end-tidal carbon dioxide gradient value being indicative of a measure of perfusion of the living organism.

### TECHNICAL FIELD

The present invention relates to quantifying pulmonary blood flow, and in particular, comparing tissue carbon dioxide (CO₂) levels and end-tidal CO₂ levels and generating a combined metric that is useful in the analysis of changes and trends in perfusion.

### BACKGROUND

In emergency room situations, mean arterial blood pressure and measures of acidosis are commonly monitored to quantify perfusion. Quantification of such clinical measurements and data related to blood flow is particularly useful in the treatment of patients requiring resuscitation from shock. Moreover, perfusion and ventilation parameters are increasingly recognized as intricately related. End-tidal CO₂ levels, in particular, are representative respiratory measures; and devices to measure end-tidal CO₂ levels, such as capnometers, have become ubiquitous in patient treatment.

### SUMMARY

Comparing tissue and end-tidal CO₂ levels may facilitate a combination of ventilation and perfusion measures into a single metric, and result in easily-detectable amplification of perfusion trends, especially considering the rise in tissue CO₂ and decrease in end-tidal CO₂ that accompanies shock. In addition, the deployment of non-invasive tissue-to-end-tidal CO₂ measures at the same location or at closely proximate locations on a patient's body may be used for both intubated and spontaneously breathing patients. Accordingly, as discussed in connection with various embodiments, the output from tissue CO₂ and end-tidal CO₂ monitors may be combined into a arterial-to-end-tidal CO₂ gradient value or set of values, thus providing a continuous, highly sensitive, integrated (i.e., including respiratory- and perfusion-based measures), non-invasive measure of perfusion status, which may be particularly useful in detecting the onset of shock as well as monitoring and quantifying the status of patients in shock.

### BRIEF DESCRIPTION OF THE DRAWINGS

For a more complete understanding of example embodiments of the present invention, reference is now made to the following descriptions taken in connection with the accompanying drawings in which:

FIG. 1 is a graphical representation of the binomial relationship observed between MAP and the PaCO₂-PetCO₂ gradient;

FIG. 2 is a graphical representation of the linear relationship observed between MAP and BD;

FIG. 3 is a graphical representation of a phase shift/latent period, in which BD inflection lags behind clinical improvement of a patient, as quantified and charted for comparison from the perspectives of comparative MAP, PaCO₂-PetCO₂ gradient, and BD measures, respectively;

FIG. 4 is a schematic view of an exemplary apparatus configured to measure tissue CO₂ and end-tidal CO₂ in accordance with various embodiments of the present invention; and

FIG. 5 is a flow chart illustrating a method for gathering tissue CO₂ data and end-tidal CO₂ data and generating a tissue CO₂-end-tidal CO₂ gradient that is indicative of a measure of perfusion of a living organism.

### DETAILED DESCRIPTION OF THE DRAWINGS

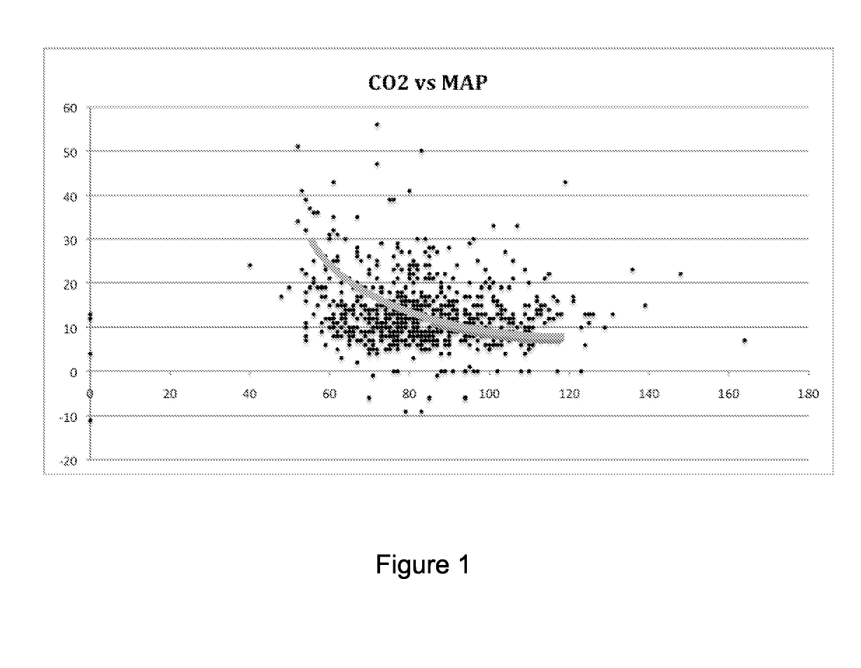

Examples and their potential advantages are understood by referring to FIGS. 1-5 of the drawings.

Mean arterial blood pressure (MAP) and measures of acidosis such as base deficit (BD) or serum lactate are widely used to quantify perfusion. Perfusion constitutes a measure of a patient's health status, and may be particularly useful in monitoring patients requiring resuscitation from shock. However, MAP and acidosis measures provide a less-than-optimal quantifiable measure for early diagnosis or for monitoring response to therapy in real time. In addition, there is currently no metric that integrates both perfusion and ventilation parameters, which are increasingly recognized as intricately related. It would be advantageous to provide a single metric that is responsive and generates real-time quantitative data regarding a patient's perfusion status, with a low degree of random variability.

A concept that combines ventilation and perfusion is that of V/Q matching, which compares lung segments with regard to each of these and is generally used in the workup of pulmonary emboli. It also appears that the concept of V/Q matching has relevance during shock, as decreasing perfusion results in an increasing proportion of lung segments that are ventilated without perfusion. This may be quantifiable by comparing the partial pressure of carbon dioxide measured from an arterial blood sample (PaCO₂) to that measured at the end of an expired breath, i.e., the end-tidal CO₂ (PetCO₂). As an increasingly greater proportion of lung segments receive ventilation without perfusion, a larger proportional discrepancy between PaCO₂ and PetCO₂ (i.e., a greater PaCO₂-PetCO₂ gradient) becomes apparent.

Moreover, end-tidal CO₂ will decrease relative to arterial CO₂ almost immediately with the onset of shock because of alterations in V/Q matching, and will recover quickly with restoration of normal perfusion. On the other hand, tissue CO₂ values rise quickly in shock and fall with therapies to reverse shock. Thus, the tissue-to-end-tidal CO₂ gradient represents a useful measure, as changes in the CO₂ gradient will be amplified by changes in perfusion.

Due to the utility of end-tidal CO₂ levels as respiratory measures, devices to measure end-tidal CO₂ levels, such as capnometers, have become ubiquitous in patient treatment. Capnometers may be placed in-line with an endotracheal tube or in the nostrils and over the mouth for spontaneously breathing patients. In addition to monitoring CO₂ levels in a patient's exhaled breath, levels of CO₂ measured at certain tissue locations provide information that is useful in diagnosing and treating patients. Although sensors for measuring tissue CO₂ at the buccal mucosa and sublingual areas may provide useful data, these may be somewhat uncomfortable especially for a patient who is awake. Measuring tissue CO₂ levels at other sites, such as the nasal septum and the concha of the ear may improve patient comfort and leverage the frequent use of other patient monitors at those sites (e.g., monitors for the measurement of SpO₂ at the concha and nasal septum, and end-tidal CO₂ adjacent to the nasal septum).

Although end-tidal CO₂ alone is commonly used as a respiratory monitor, it is not commonly considered an indicator of perfusion, particularly in the absence of a reference (tissue or arterial CO₂ data). However, data are emerging regarding the use of tissue CO₂ as a measure of shock. Although it is reported that although the corneum stratum (outer skin layer) is extremely thin and thus amenable to obtaining tissue CO₂ measurements in young patients, obtaining such tissue CO₂ measurements in adults or older children may not be as reliable, due to difficulties related to beaming infrared light through the outer layer of a patient's skin. Moreover, although reports comparing arterial-to-end-tidal CO₂ have appeared sporadically, such comparisons depend on periodic acquisition of an arterial blood gas, which is not continuous and somewhat invasive. Thus, measuring CO₂ at locations other than mucous membranes may pose challenges.

To illustrate the point made above, regarding tissue-to-end-tidal CO₂ gradient, a comparison was performed between MAP, BD, and the PaCO₂-PetCO₂ gradient with regard to the ability of each of these measures to accurately reflect perfusion status in a population of critically ill and injured intensive care unit (ICU) patients. Accuracy was assessed by measuring both the responsiveness to changes in clinical perfusion status as well as the random variability for each metric.

### EXPERIMENTAL METHOD AND RESULTS

### Patient Inclusion Criteria/Methodology

An observational study was performed on a sample of patients at a health care facility and data were gathered. In connection with the data collection, nursing staff was instructed to document PetCO₂ simultaneous to arterial puncture for all patients for whom capnography was being used and an arterial blood gas was being obtained. Capnography was recommended for all patients undergoing endotracheal intubation as both a mechanism for continuous confirmation of tube placement and to help refine ventilation settings.

### Data Collection

All clinical data, including vital signs, laboratory data, continuous infusions (medication and rate), and clinical measurements including PetCO₂ were recorded in a unit-specific electronic database. For the purpose of this analysis, the following specific data were abstracted electronically: MAP, arterial blood gas data (PaO₂, PaCO₂, BD, pH), ventilator settings, serum lactate, serum electrolytes, and pressor infusions. The hospital electronic patient care record was abstracted for the following clinical data: demographics, admission and discharge diagnoses, comorbitidies, blood transfusions, and procedures including major operations.

### Data Analysis

Demographic data were analyzed descriptively. The general relationships between MAP, BD, and PaCO₂-PetCO₂ gradient were explored using polynomial regression. Data from all eligible patients were included. The goodness-of-fit was quantified using r-values, and the overall relationships were evaluated using the resultant regression curves.

The main analysis considered the ability of each parameter to accurately reflect the clinical course of each patient over time with minimal random fluctuation in serial readings. Only patients with at least 10 simultaneous arterial blood gas/PetCO₂ measurements were included. The hemodynamic course for each patient was defined using a series of “epochs” defined by vital signs (MAP, heart rate), clinical events (blood transfusions, operative interventions, discharge from the ICU or death), and pressors. Each epoch was defined as representing a period in which patients were getting sicker or getting better (i.e., deterioration in a patient's condition, or recovery) based on the above parameters. Each patient could have anywhere from one to five epochs.

Polynomial regression was then used to generate a best-fit curve charting the MAP, BD, and PaCO₂-PetCO₂ gradient data over time. The equation degree was based upon the number of epochs defined for each patient (i.e., a 1^{st} degree equation was used for a single epoch, 2^{nd} degree equation for two epochs, 3^{rd} degree equation for three epochs, etc.). The responsiveness of each metric was defined by calculating the number of epochs accurately identified as deteriorating or recovering, based on the slope of the curve. The random variability for each measurement was quantified by calculating the mean r-value across all patients. The r-value for each patient was defined as the mean of the absolute values for r-values determined for each epoch.

### RESULTS

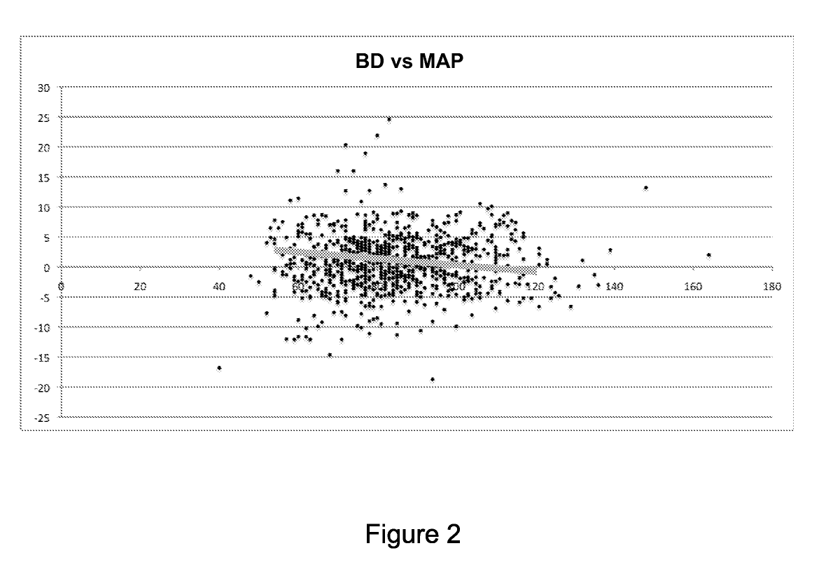

Data from a total of 168 patients with 1,082 simultaneous arterial blood gas and PetCO₂ measurements were included in this analysis. Polynomial regression revealed moderate correlation between MAP and both BD as well as the PaCO₂-PetCO₂ gradient. A binomial relationship between MAP and the PaCO₂-PetCO₂ gradient was observed, with a rise in this gradient indicating hypotension but with no change with normo- or hypertension. FIG. 1 is a graphical representation of the binomial relationship between MAP and the PaCO₂-PetCO₂ gradient. Conversely, a linear relationship was observed between MAP and BD, as illustrated in FIG. 2, with lower BD values for hypertension as opposed to normo- or hypotension. Accordingly, the PaCO₂-PetCO₂ gradient provided a more physiological model than BD.

A total of 27 patients with 63 epochs were included in the temporal analysis. Mean r-values were similar for the PaCO₂-PetCO₂ gradient (r=0.59) and BD (0.58), which were both higher than the r-value for MAP (r=0.37). The “smoothness” of the regression lines/curves was defined by the mean of absolute r values. The r-value corresponding to MAP thus indicated a much higher level of variability from point-to-point. It was then determined whether the PetCO₂ line/curve and the BD curve correctly characterized whether a patient was getting sicker (positive slope for PetCO₂, negative slope for BD) or recovering (negative slope for PetCO₂, positive slope for BD), for each epoch. The r-value corresponding to MAP thus indicated a much higher level of variability from point-to-point. Correct classifications were made in 97% of epochs using the PaCO₂-PetCO₂ gradient but only 51% of epochs using BD. The majority of BD misclassifications involved a phase shift or latent period, in which the base deficit inflection lagged behind the clinical improvement of the patient. These data indicate that measuring the CO₂ gap is much faster (and thus more accurate) than traditional measures of acidosis (base deficit, with the same likely the case for lactate) and “smoother” than MAP. This indicates that the CO₂ gap could be used to guide clinical decisions in real time, whereas currently used parameters cannot.

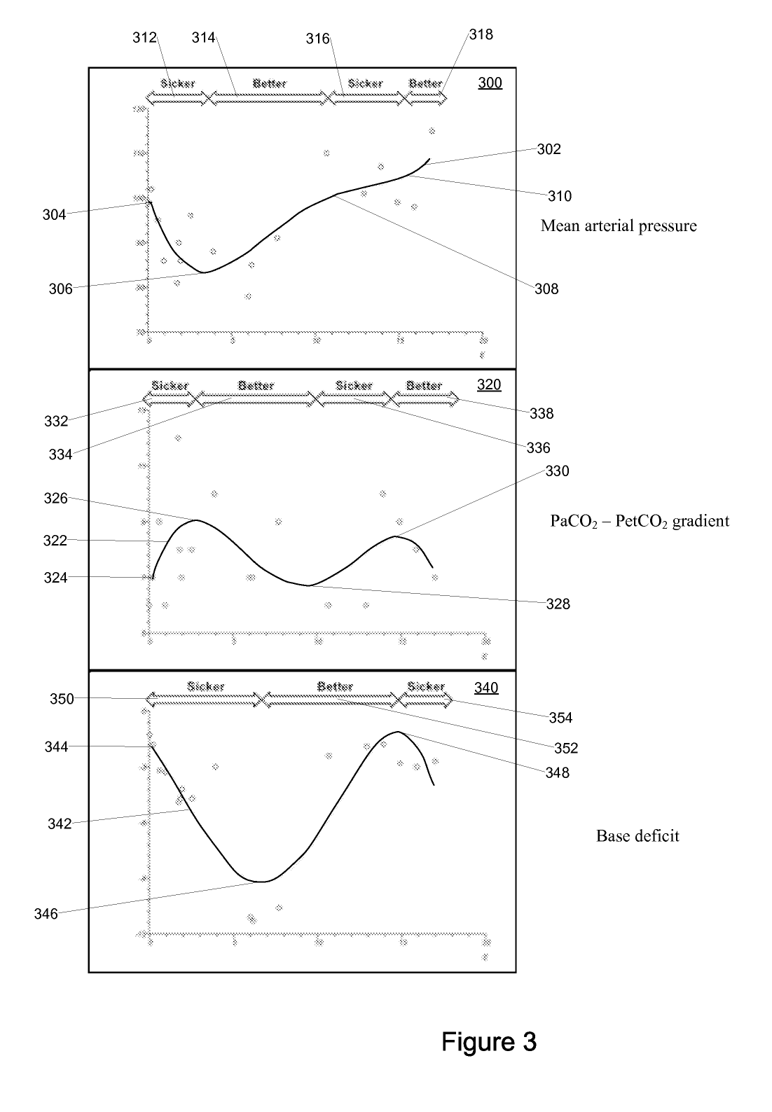

FIG. 3 illustrates this phenomenon as a graphical comparison of the change in each of these measures over time, and the correlation of epochs to inflection points on the various curves. More specifically, graph 300 illustrates a curve 302 fitted to measured mean arterial pressure (MAP) values over time, with start point 304 and inflection points 306, 308, 310, each corresponding to the start of a respective epoch 312, 314, 316, 318. Similarly, graph 320 illustrates a curve 322 fitted to measured PaCO₂-PetCO₂ gradient values over time, with start point 324 and inflection points 326, 328, 330, each corresponding to the start of a respective epoch 332, 334, 336, 338. Graph 340 illustrates a curve 342 fitted to measured BD values over time, with start point 344 and inflection points 346, 348, each corresponding to the start of a respective epoch 350, 352, 354.

### CONCLUSION

Polynomial regression analyses demonstrated the potential usefulness of the PaCO₂-PetCO₂ gradient in monitoring perfusion status. As illustrated by a comparison between graphs 320 and 340, the PaCO₂-PetCO₂ gradient values were more accurate than BD values in reflecting clinical course in real time, with a latent period associated with BD clearly shown. In addition, curve 322 fitted to the PaCO₂-PetCO₂ gradient values was “smoother,” as seen in graph 320, with less random variability than the curve 302 fitted to the MAP values, illustrated in graph 300.

Due to their inherent characteristics and relationships with other measures of bodily health and function, combining tissue CO₂ and PetCO₂ measurements results in useful data and information for patient treatment. PetCO₂ is the most reactive measure of blood flow through the lungs, but PetCO₂ is also affected by PaCO₂, which rises and falls with various tidal volume and ventilation flow rate values. Based on the model that gives rise to the experimental results above, PaCO₂ will fall between tissue CO₂ and PetCO₂. As a result, the goal of therapies may thus be to narrow the CO₂ gap and bring tissue CO₂ and PetCO₂ closer. In other words, tissue CO₂ may serve as a reference for PetCO₂ to determine whether the PetCO₂ values are low due to hypoperfusion or overventilation.

Based on the above experimental demonstration, the PaCO₂-PetCO₂ gradient demonstrates an improvement over current techniques and values (i.e., MAP and BD) for measuring and monitoring perfusion status. Moreover, end-tidal CO₂ is increasingly recognized as reflecting pulmonary blood flow, and a small but compelling body of literature exists to support the concept of the arterial-to-end-tidal CO₂ gradient as an improved measure of shock, as this adjusts for the possibility of arterial hyper- or hypocapnia. In addition, as noted above, tissue CO₂ levels also correlate well with shock and shock-related mortality. Comparing tissue CO₂ and end-tidal CO₂ levels results in advantages such as: i) the potential amplification of perfusion trends with the rise in tissue CO₂ and decrease in end-tidal CO₂ that accompanies shock; and ii) the ability to combine ventilation and perfusion measures into a single metric. In addition, tissue-to-end-tidal CO₂ measures in embodiments may be non-invasive and could potentially be used with both intubated and spontaneously breathing patients. Embodiments that include non-invasive sensors for measuring these parameters may thus be utilized with patients known to be critically ill, as well as with potentially ill patients as a screening tool. Thus, embodiments may provide a continuous, highly sensitive, integrated (relying on respiratory and perfusion measures), non-invasive measure of blood perfusion, which may be useful in quantifying and monitoring levels of shock in patients.

Various exemplary embodiments of systems and methods will now be discussed for the measurement of tissue CO₂ and end-tidal CO₂, as well as the comparison of these respective measures and the calculation of a tissue-to-end-tidal CO₂ gradient or gap. Embodiments may be non-invasive, and may leverage and combine existing technology to measure and compare tissue CO₂ and end-tidal CO₂ levels. For example, and as noted above, capnometers, which are useful for measuring end-tidal CO₂, are ubiquitous and can either be positioned in-line with an endotracheal tube in intubated patients, or in the nostrils and over the mouth for spontaneously breathing patients. Capnometers and other conventionally-used devices such as cannulae (with as-needed modifications), for example, may be utilized to measure end-tidal CO₂ values in patients for analysis and quantification into a tissue-to-end-tidal CO₂ gradient value(s) in accordance with embodiments.

Tissue CO₂ monitoring, on the other hand, may be performed in any well-perfused location on a patient's body, such as mucous membrane sites including the nasal septum, buccal mucosa and sublingal area, for example. Similarly to the use of capnometers and other such devices for measuring end-tidal CO₂ values, various types of conventional tissue CO₂ measuring sensors may be used in embodiments, such as sensors incorporating electrochemical or optical technology (both electrochemical or optical technology-based sensors are used in various biosensors throughout the medical field). However, as discussed above, applying tissue CO₂ sensors to certain areas such as the buccal mucosa and sublingal areas may be uncomfortable for a patient who is awake. Alternative sites for measuring tissue CO₂ levels include the nasal septum and concha of the ear, where measurements may be taken utilizing device such as a pulse oximeter, for example. In embodiments, the tissue CO₂ and end-tidal CO₂ measuring sensors may be integrated into a unitary device, or they may be completely separate and attached to different areas of a patient's body.

Due to better tolerability for patients, the nasal septum may function as an attractive target site to measure tissue CO₂. Although perfusion may be maintained to the nasal septum in low flow states, as with other sites on a patient's face, a tissue CO₂ sensor in a patient's nose may be better tolerated than one in his or her mouth, particularly since nasal prongs and a mouth “scoop” may already be in place in such as nasally-oriented device such as in a device for end-tidal CO₂ measurements in non-intubated patients. Moreover, in an embodiment, tissue CO₂ measurement may be performed at the same site (i.e., in proximity to the nasal septum, as discussed), by utilization of tissue and end-tidal CO₂ measurement sensors that are separate devices or integrated into a unitary device. Non-invasive measurement of tissue and end-tidal CO₂ values may thus be facilitated without employing additional sensors, i.e., by utilizing and leveraging sensors being utilized for other measurements. Although example embodiments are described as configured for the performance of such measurements at the same location on a patient's body, such as in the vicinity of the nasal septum, for example, any combination of a tissue CO₂ sensor and an end-tidal CO₂ measurement sensor, whether integrated and unitary, or separable, may thus facilitate measurement of values used in the calculation of the tissue-to-end-tidal CO₂ gradient value(s).

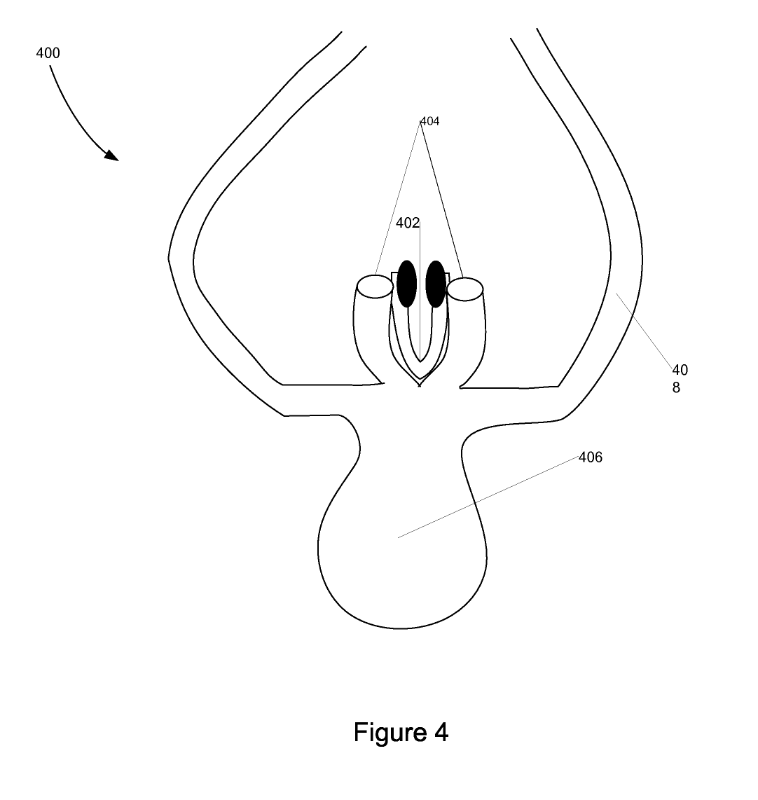

FIG. 4 illustrates an exemplary embodiment of a device 400 for the noninvasive measurement of tissue CO₂ and end-tidal CO₂. In particular, the device 400 illustrated in FIG. 4 may include a tissue CO₂ sensor 402 and end-tidal CO₂ sensors 404, 406. The tissue CO₂ sensor 400 may be configured to be maintained substantially about a patient's nasal septum, while the end-tidal CO₂ sensors 404, 406 may be configured in a mask-like structure 408 to capture and facilitate the measurement of CO₂ exhaled from the nasal cavity and the mouth. Of course, any number and type of appropriate sensors may be deployed for accurate and effective measurement and gathering of tissue CO₂ and end-tidal CO₂ data. Measurement of tissue CO₂ levels may thus be facilitated by proximity of the tissue CO₂ sensor 400 to tissue surrounding a patient's nasal septum, and the measurement and collection of CO₂ levels in a patient's exhaled breath may be facilitated by end-tidal CO₂ sensors 404, 406. Such a device 400 may provide co-location, or proximate location of the tissue and end-tidal CO₂ measurement sensors in a non-invasive configuration, thus improving patient tolerability, particularly in patients who are awake. Moreover, such co-location may be advantageous for those administering treatment to the patient, or measuring the relevant data. Co-location may reduce the number of separate sensors and devices to be attached to a patient, and make them easier to keep track of, particularly when a large number of other sensors and devices are attached.

For some of the reasons discussed above, among others, a patient's ear concha may serve as another potential site for measuring and recording tissue CO₂. Advantages of this area may include good perfusion in low-flow states and the possibility of an existing sensor at that site for gathering and recoding clinical data that is useful in connection with other patient measures (with use of a pulse oximeter, for example). However, as discussed in greater detail herein, the corneum stratum (outer layer of the skin) may pose challenges in accurately and reliably measuring tissue CO₂ levels in non-infant patients through the use of such devices.

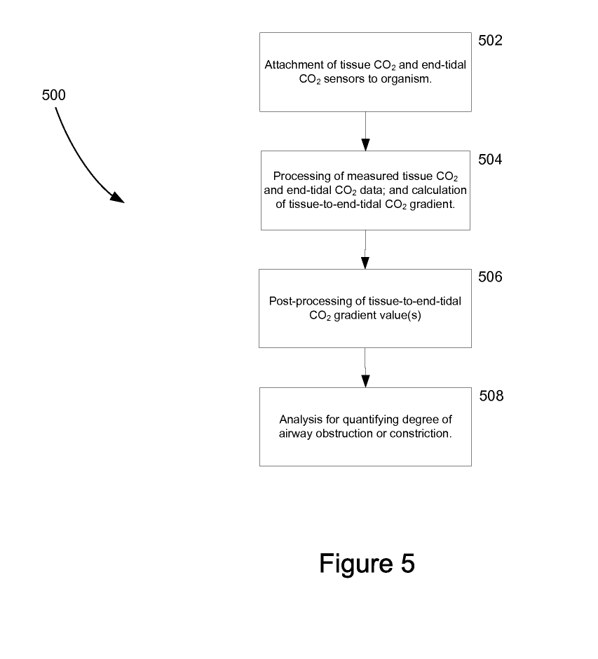

Having discussed various components of a system for taking certain clinical measurements pertaining to a patient, for use in the computation of tissue-to-end-tidal CO₂ gradient values, an exemplary method of implementation will now be discussed. Referring now to FIG. 5, there is illustrated a process 500 including the measurement and recording of data pertaining to a living organism such as a human patient, for example, for the calculation of tissue-to-end-tidal CO₂ gradient values. At box 502, sensors for measuring and capturing tissue CO₂ levels and end-tidal CO₂ levels may be attached to the organism. As discussed above, tissue CO₂ levels and end-tidal CO₂ levels of a living organism may be measured through various sensors and devices, which may be non-invasive, in embodiments. In various embodiments, such sensors and devices for measuring end-tidal CO₂ may be attached to one or more of a patient's nostrils, mouth, or an endotracheal tube attached to the patient; while sensors and devices for measuring tissue CO₂ may be attached to a patient's buccal mucosa, sublingual, nasal septum or ear concha regions. Measurement of end-tidal CO₂ level data may then be performed in an endotracheal tube attached to the organism, nostrils of the organism, and/or the mouth of the organism. Moreover, the measurement of such data may be facilitated by devices such as a capnometer for measuring end-tidal CO₂ values and a pulse oximeter for measuring tissue CO₂ levels. Also, these measurements may be conducted simultaneously, or separated by some time differential. At box 504, measured tissue CO₂ levels and end-tidal CO₂ levels of the organism may be operated on or processed in the calculation of at least one tissue-to-end-tidal CO₂ gradient value. The tissue-to-end-tidal CO₂ gradient may thus be based on the measured tissue CO₂ and end-tidal CO₂ values. As discussed above, such a gradient may be indicative of a measure of perfusion in the organism and this measure of perfusion may be utilized in diagnosing and measuring shock in an organism, such as a patient undergoing medical treatment or seeking medical attention.

At box 506, various types of parallel- or post-processing of calculated tissue-to-end-tidal CO₂ gradient values may occur, especially as may be useful for analysis of this gradient, or gap. For example, because end-tidal CO₂ varies from breath-to-breath, but tissue CO₂ reacts more slowly (over minutes), the output CO₂ gradient values may be represented as a 60-120 second moving average. Moreover, further processing and analysis steps may yield quantification of the degree of airway obstruction or constriction as affecting the tissue-to-end-tidal CO₂ gradient value(s), as illustrated at box 508. For example, since the presence of reactive airway disease (“RAD,” e.g., COPD, asthma) may affect tissue-to-end-tidal CO₂ gradient value(s), measurements and data regarding the effects of RAD may be incorporated into the tissue-to-end-tidal CO₂ gradient calculations to improve the accuracy thereof. Such further measurement, processing and analysis may be particularly effective and accurate in spontaneously breathing patients. Conversely, calculating the most accurate tissue-to-end-tidal CO₂ gradient value(s) possible may thus also facilitate the ability to quantify the degree of airway obstruction or constriction continuously and automatically (particularly as an improvement over existing methods that employ peak flow meters).

While various embodiments of the present invention have been described above with regard to particular contexts/implementations, it should be understood that they have been presented by way of example only, and not of limitation. Likewise, the various diagrams may depict an example architectural or other configurations for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

Moreover, various embodiments described herein are described in the general context of method steps or processes, which may be implemented in one embodiment by a computer program product, embodied in a computer-readable memory, including computer-executable instructions, such as program code, executed by computers in networked environments. A computer-readable memory may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes. Various embodiments may comprise a computer-readable medium including computer executable instructions which, when executed by a processor, cause an apparatus to perform the methods and processes described herein.

Furthermore, embodiments of the present invention may be implemented in software, hardware, application logic or a combination of software, hardware and application logic. The software, application logic and/or hardware may reside on a client device, a server or a network component. If desired, part of the software, application logic and/or hardware may reside on a client device, part of the software, application logic and/or hardware may reside on a server, and part of the software, application logic and/or hardware may reside on a network component. In an example embodiment, the application logic, software or an instruction set is maintained on any one of various conventional computer-readable media. In the context of this document, a “computer-readable medium” may be any media or means that can contain, store, communicate, propagate or transport the instructions for use by or in connection with an instruction execution system, apparatus, or device, such as a computer. A computer-readable medium may comprise a computer-readable storage medium that may be any media or means that can contain or store the instructions for use by or in connection with an instruction execution system, apparatus, or device, such as a computer. In one embodiment, the computer-readable storage medium is a non-transitory storage medium.

Although various aspects of the invention are set out in the independent claims, other aspects of the invention comprise other combinations of features from the described examples and/or the dependent claims with the features of the independent claims, and not solely the combinations explicitly set out in the claims.

The foregoing description of examples has been presented for purposes of illustration and description. The foregoing description is not intended to be exhaustive or to limit examples of the present invention to the precise form disclosed, and modifications and variations are possible in light of the above teachings or may be acquired from the practice of various examples. The examples discussed herein were chosen and described in order to explain the principles and the nature of various examples and its practical application to enable one skilled in the art to utilize the present invention in various examples and with various modifications as are suited to the particular use contemplated. The features of the examples described herein may thus be combined in all possible combinations of methods, apparatus, modules, systems, and computer program products.

What is claimed is:

1. A method, comprising:
measuring tissue carbon dioxide levels of a living organism;
measuring end-tidal carbon dioxide levels of the living organism; and
calculating at least one tissue-to-end-tidal carbon dioxide gradient value based on the measured tissue carbon dioxide levels and the measured end-tidal carbon dioxide levels, the tissue-to-end-tidal carbon dioxide gradient value being indicative of a measure of perfusion of the living organism.

2. The method of claim 1, wherein the measurement of the tissue carbon dioxide levels is non-invasive and performed in at least one of: a buccal mucosa region of the living organism, a sublingual region of the living organism, a nasal septum region of the living organism, and an ear concha region of the living organism.

3. The method of claim 1, wherein the measurement of the end-tidal carbon dioxide levels is non-invasive and performed in at least one of: an endotracheal tube attached to the living organism, nostrils of the living organism, and a mouth of the living organism.

4. The method of claim 1, wherein the non-invasive measurement of the end-tidal carbon dioxide levels is performed via a capnometer.

5. The method of claim 1, wherein the non-invasive measurement of the tissue carbon dioxide levels is performed via a pulse oximeter.

6. The method of claim 1, further comprising:
performing post-processing on the tissue-to-end-tidal carbon dioxide gradient value.

7. The method of claim 1, the measure of perfusion being indicative of a level of shock experienced by the organism.

8. A system, comprising:
at least a first sensor configured to non-invasively measure tissue carbon dioxide levels in a human being;
at least a second sensor configured to non-invasively measure end-tidal carbon dioxide levels in the human being; and
a calculating apparatus comprising at least one processor and at least one memory including computer program code, the at least one memory and the computer program code configured to, with the at least one processor, cause the calculating apparatus to calculate at least one tissue-to-end-tidal carbon dioxide gradient value based on the measured tissue carbon dioxide levels and the measured end-tidal carbon dioxide levels, the tissue-to-end-tidal carbon dioxide gradient value being indicative of a level of perfusion of the living organism.

9. The system of claim 6, wherein the first sensor comprises a capnometer.

10. The system of claim 6, wherein the second sensor comprises a pulse oximeter.

11. The system of claim 6, wherein the at least one memory and the computer program code are further configured to, with the at least one processor, cause the calculating apparatus to perform post-processing on the tissue-to-end-tidal carbon dioxide gradient value.

12. The system of claim 6, the measure of perfusion being indicative of a level of shock experienced by the organism.
